(19) United States
(12) Patent Application Publication
(10) Pub. No.: US 2002/0103435 A1
(43) Pub. Date: Aug. 1, 2002

(54) Ultrasonic monitoring of bone density with diet feedback

(76) Inventor: James R. Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/035,629

(22) Filed: Oct. 26, 2001

Related U.S. Application Data
(60) Provisional application No. 60/243,544, filed on Oct. 26, 2000.

(51) Int. Cl. A61B 8/00

(52) U.S. Cl. 600/439

(57) ABSTRACT

A system for monitoring bone density includes a monitor employing ultrasonic transducers adapted to be supported in contact with the user's body. The bone density information derived is provided to a personal digital assistant including a display, including means for entering the information into the Internet. A remote computer connected to the Internet includes a program for analyzing the data and providing feedback to the user which is displayed on the personal digital assistant. The user may also record information relating to food consumption and the feedback information may contain dietary recommendations.

### RELATED APPLICATION

This application claims priority of U.S. Provisional Patent Application No. 60/243,544 filed Oct. 26, 2000 and is incorporated herein by reference.

### FIELD OF THE INVENTION

This invention relates to monitoring bone density and calcium content of bone structures, as well as assisting adherence to a healthy diet.

### BACKGROUND OF THE INVENTION

Often, as a result of maintaining a low calorie diet for the purposes of losing weight, the calcium content of an individual's bones tend to decrease. It is important to monitor this condition so that an individual's bone strength will not deteriorate to a dangerous level.

It has been found that it is very important to monitor this bone, or calcium condition, so as to take appropriate steps to correct the condition if it approaches a dangerous level. Low calcium levels may lead to osteoporosis and bone fractures. One means of correcting this deficiency of calcium is by increasing the amount of exercise of the patient. Of course, another way of preventing this calcium deficiency is by taking internal medicine, calcium supplements, or altering one's dietary consumption.

Ultrasonic devices have been utilized for measuring the physical properties of various components, such as bones and muscles, and examples of such devices are disclosed in U.S. Pat. No. 3,477,422 granted to J. M. Jurist, Jr. et al. on Nov. 11, 1969; U.S. Pat. No. 3,345,863 granted to E. A. Henry et al. on Oct. 10, 1967; U.S. Pat. No. 3,847,141 granted to James M. Hoop et al. on Nov. 12, 1974; U.S. Pat. No. 5,564,423 granted to Ruggero Mele et al. on Oct. 15, 1996; and U.S. Pat. No. 5,218,963 granted to Richard B. Mazess on Jun. 15, 1993. The devices described in the above-mentioned references calculate the bone density of a particular area by comparing the input and output velocities of ultrasonic waves traveling through the bone. Further, these references disclose that the bone density of one area of the body can be used to approximate that of other areas.

It would be desirable to provide a device which measures and calculates bone density and incorporates this data in an analysis of an individual's diet and overall health. The present invention accomplishes this end.

Body fat analyzing scales are sold commercially, for example by the Tanita company of Japan. A person may lose calcium from their bones during e.g. weight control programs, pregnancy, aging processes, disease, malnutrition, and the like. It is advantageous to provide a modified scale having the capability of monitoring bone density.

### SUMMARY OF THE INVENTION

The present invention operates to assist individuals in maintaining their health while attaining their dietary goals. Information relevant to the individual's health is inputted into the computing device of the present invention, stored, and analyzed, and the computing device returns suggestions and feedback to the user.

The user inputs, by means well known in the art, details regarding all consumption of food. A preferred embodiment of the present invention also allows the input of data relating to the purchase of food for ease of use. This information is stored in a database and remains available for future analysis.

The user may also enter data regarding herself or another individual. This individual data may include age, gender, health problems, height, weight, and other factors pertinent to the maintenance of a diet and an individual's overall health. The individual data is stored by the computing device of the present invention in a personal health database, and may be used by the computing device for the purpose of analysis, as discussed below.

The preferred embodiment of the present invention further possesses at least one bone density sensor. The bone density sensor(s) may be of the type which utilize ultrasonic waves. Such an ultrasonic sensor is positioned on a suitable portion of the user's body. Said portion of the user's body may be one of a large number of locations, such as the waist, chest, wrist, foot, or limb. Preferably, the ultrasonic sensor communicates data relating to the bone density of said portion of the user's body to a personal digital assistant (PDA). The PDA analyzes this data in a manner well known in the art to determine overall bone density. This information is then stored in a database for future reference.

The above-mentioned inputs are all stored in the RAM of the computing device of the present invention. Upon entry of a food intended to be purchased or consumed or measurement of bone density, the computing device of the present invention may analyze the user's choice of food or the user's bone density. The computing device then provides feedback to the user based on any combination of food choice, bone density calculation, and other stored data. This feedback may include the nutritional information of a food choice as well as information regarding the overall progress of the individual's diet and the individual's overall health. This feedback may further include suggestions relating to the user's exercise requirements and necessity for greater calcium consumption.

Further, a preferred embodiment of the present invention includes a smart card, or other portable data storage device. This data storage device allows the user to transfer data to and from the PDA.

In a further preferred embodiment of the present invention, the computing device of the present invention may create graphs, tables, and charts based on the data it has stored. These visual aids may then be printed in the manner described above, or displayed by the PDA.

In yet another preferred embodiment of the present invention, the computing device is capable of communicating with other computers, by such means as the Internet or a modem. In this manner, an individual's physician may monitor the progress and health of the individual without the need for an office visit.

Any information stored in the computing device of the present invention may be printed by conventional means. A printing device may be provided at locations such as grocery stores, physicians' offices, restaurants, and the user's home. Additionally, a preferred embodiment can be placed in electrical communication with a personal computer to facilitate the printing of data at the user's home. Connection to a personal computer may also make data entry into the computing device of the present invention easier.

Other objects, advantages and applications of the present invention will be made apparent by the following detailed description of several embodiments. The description makes reference to the accompanying drawings in which:

### BRIEF DESCRIPTION OF THE DRAWINGS

FIG. 1 is an illustration of a personal digital assistant used in connection with the present invention;

FIG. 2 is a schematic illustration of an ultrasonic bone density monitor formed in accordance with the present invention mounted on the waist of a user and in communication with a personal digital assistant of the type illustrated in FIG. 1;

FIG. 3 illustrates the manner of insertion or removal of memory modules from the bone density monitor of FIG. 2;

FIG. 4 is a schematic diagram of an ultrasonic bone density monitor formed in accordance with the present invention;

FIG. 5 is an illustration of a wrist mounted bone density monitor;

FIG. 6 is a top view of a weight scale containing ultrasonic transducers for determining the bone density, body fat percentage and the like of a person using the scale;

FIG. 7 is a schematic diagram of a system connecting a bone density monitor to a remote computer system to provide other health related information to the remote computer; and

FIG. 8 is a schematic diagram of a system for transferring information from a bone density monitor to a desktop computer, via a personal digital assistant, and from the desktop computer to a remote computer system.

### DETAILED DESCRIPTION OF THE PREFERRED EMBODIMENTS

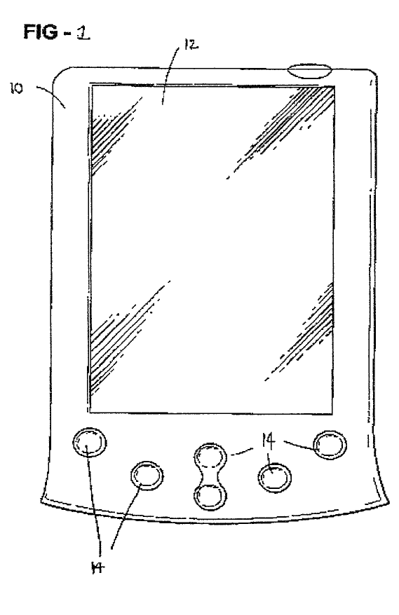

FIG. 1 illustrates a portable computer or personal digital assistant (PDA) which is used in embodiments of the present invention. The PDA 10 includes a display screen 12 which may be a liquid crystal display. A plurality of buttons 14 are provided to enable the user of the present invention to input various data. The user may also input data by using a stylus, touch pad, voice recognition software, an electronic pen, a roller, or other data input mechanism. The portable computing device of the present invention may be embodied in any one of a number of different types of personal digital assistants (PDAs). PDAs are becoming increasingly popular among all types of consumers. PDAs include the Palm Pilot family of products, Microsoft Windows CE-based handheld computers, and many others. For purposes of this application, PDAs are defined to include all types of portable computing devices including palm size and handheld computers, portable and cellular phones, pagers, and any other devices which are portable and possess some computing power.

A PDA may be adapted to contain ultrasonic transducers for the purpose of bone density monitoring. An ultrasonic monitoring module may be adapted to form a plug-in accessory for a PDA.

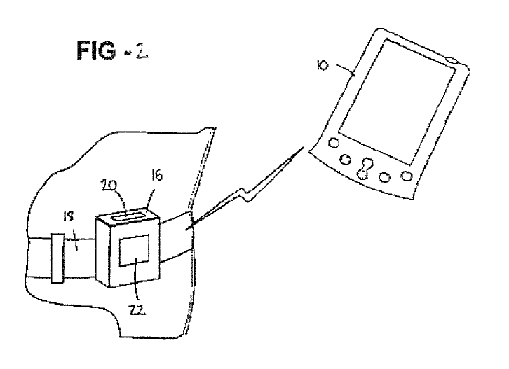

FIG. 2 shows an ultrasonic bone density monitor 16 mounted on belt 18 around the waist of a user. A chest strap or wrist strap may also be used. The monitor 16 has a display 22 and interface slot 20. Monitor 16 is preferably in wireless communication with PDA 10. IR communication, cables, or a docking interface can also be used to transfer data between the monitor and the PDA.

The display 22 is used to indicate operation of the device, and may comprise an indicator light, an alphanumeric display, or a graphical display. The PDA 10 may be used to display data, e.g. received from the monitor 16 using wireless communication methods (IR or Bluetooth radio protocol for example) or using a cable. The interface slot is used to accept memory modules for the purpose of data transfer to another device.

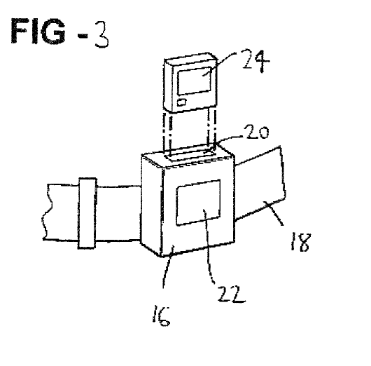

FIG. 3 shows an ultrasonic monitor 16 showing the removal or insertion of a memory module 22 into slot 20. In another embodiment, module 16 may have a connector to a cable for data transfer to another device.

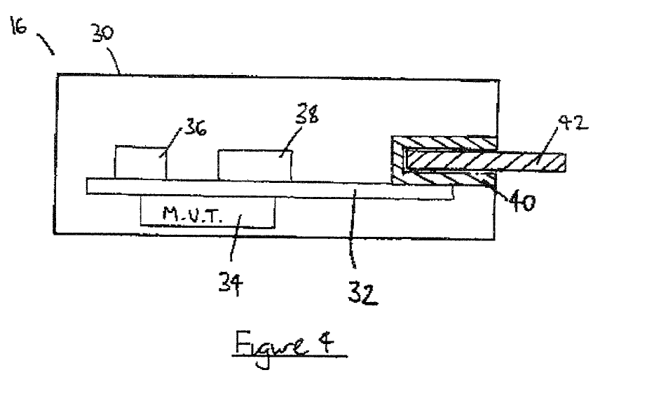

FIG. 4 shows one embodiment of an ultrasonic bone density monitor. The monitor 16 has housing 30 containing circuit board 32. A micromachined ultrasonic transducer (MUT) 34 is mounted on the board 32. Transducer control circuit 36 and memory 38 are also mounted on the board 32. A memory module 42 is shown inserted into slot 30, so as to allow data to be transferred from memory 38 to module 42. The memory card is then removed so as to allow data transfer to a PDA or other computing device.

The device may be held close to the skin on a strap, or attached to the skin using an adhesive layer and/or gel, or placed in a holder attached to the skin, for example a plastic clip adhered to the skin. A plurality of sensors 16 may be disposed around a strap or belt, or mounted around the body on skin-mounted clips. Bone density can be measured using ultrasound reflected from bones, attenuated by bones, or using the time-of-flight of ultrasound pulses through bones. Preferably, the ultrasound transducer is a micromachined ultrasonic array, an array of elements micromachined on a silicon (or other suitable material) substrate.

Using a single transducer 16, bone density may be obtained from analysis of the ultrasonic signal reflected from the bone, or signals reflected from an ultrasonic reflector (e.g., an interface) the opposite side of a bone. A reflection ultrasound method is described in U.S. Pat. No. 5,038,787 by Antich et al. For embodiments using multiple transducers, transmissions through the bone or the angular dependence of ultrasonic reflection may be studied. A transmission method is described in U.S. Pat. No. 4,361,154 to Pratt.

Images or parameters related to bone density may be displayed, e.g. on a PDA. The PDA may have a built in ultrasonic transducer. The transducer may also be part of an accessory which docks, interfaces, plugs into, or otherwise is in contact or communication with the PDA. An advantage of this embodiment is that the processing power, user interface, and display functionality of the PDA is used to show and analyze data obtained using the monitor. The PDA can store data over time for analysis and display of trends. The PDA can also be used for diet logging, as discussed in more detail below.

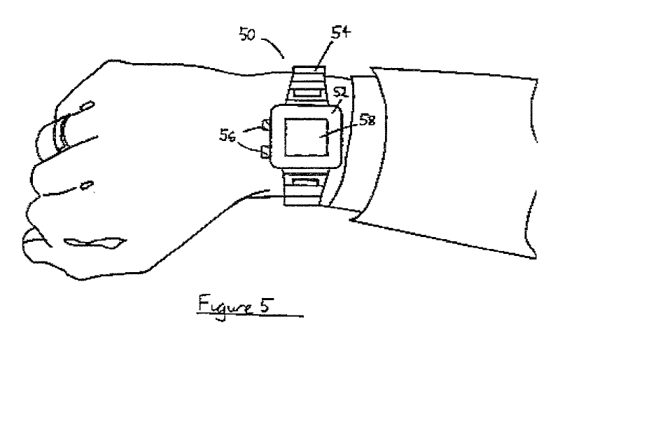

FIG. 5 shows a wrist mounted monitor 50 having a housing 52, a strap 54, a display 58 on the front of the housing 52, and buttons 56. Ultrasonic transducers are contained within the housing 50, preferably towards the rear of housing 52 so as to be in close proximity to the wrist of the user. A gel pad may be used to improve the acoustic coupling between the transducer and the user's wrist. Transducers may also be mounted on the strap 42. Buttons 56 are used to change the operating mode of the device, initiate ultrasonic scanning, set the time shown on display 58, enter data, and the like. For example, the device may be configured as a combined wristwatch and bone density monitor, and in other embodiments may also comprise the functionality of glucose monitor, radio, wireless phone, videophone, video recorder, PDA, digital camera, computer, plethysmograph, or oximeter.

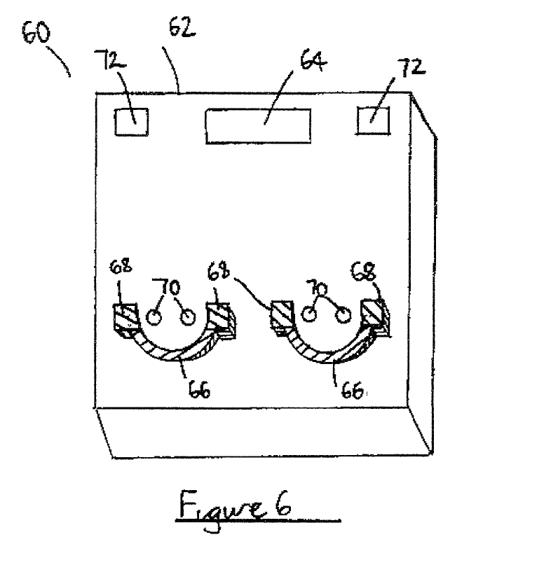

FIG. 6 shows a top view of a scale shown generally at 60, having a housing 62 which contains a conventional weight-measuring mechanism. The top side of the housing has a display 64, toe-operated switches 72, heel location guides 66 protruding upwardly from the top surface of the housing 62, ultrasonic transducers 68 located so as to transmit ultrasonic radiation through the heel of a person standing on the scale, and electrodes 70 located so as to contact the feet of a person standing on the scale.

In use, a person stands on scale 60, and their weight is displayed on display 64. By operating a switch 72, the person can switch to body fat analysis, in which a conventional four-electrode bioimpedance method is used to determine the person's body fat percentage, which is then shown on display 64. Heel guides, according to the present invention, improve the reproducibility of bioimpedance measurements. According to the present invention, bone density can be determined using ultrasound attenuation, ultrasonic pulse propagation time, reflection, broadband attenuation spectra, or angular-dependent reflection properties of the heel bone (or using other density-related properties of a bone such as mechanical oscillation, ultrasonic image properties, and the like). In alternative embodiments, a shaped depression is used to locate the foot. The display 64 may display weight, body fat percentage, and bone density simultaneously. Switches 72 may also be used to identify the user, enter height, and the like.

In another embodiments, scale 60 does not have switches 72, and displays all determined parameters on display 64. These may be displayed together or in a time sequential manner. In another embodiment, bioimpedance measurement functionality is not provided by the scale. In another embodiment, hydration levels of the body are determined using bioimpedance analysis.

It is useful to monitor body fat and bone density together, particularly during weight loss programs.

An ultrasonic bone density monitor may also be implanted into the body, for example into bone material, or into a tooth crown. A bone density monitor may also be held close to the body using a strap around the chest, ankle, wrist, finger, toe, etc., a ring, ornamentation, button, and the like, or contained in clothes or shoes. Shoes containing ultrasonic transducers can be used to monitor the heel bone, with wireless transmission to a PDA.

Bone Density Scanning With Diet Logging in Health Management System

Diet logging software may be used to monitor the nutritional intake of the user. A sufficient calcium intake is important for maintaining a healthy level of bone density and strength. A user may use a PDA, wrist mounted device, or other device to scan barcodes on packages. The UPC (universal product code) may be used to create a diet log and extract nutrient, vitamin and mineral contents from a database.

If intake of any dietary component is too low, supplements may be recommended. A program of sustained bone strength or density increase development may be devised. The user is supplied with calcium supplements and a bone density monitor. Records of bone density are made over time, along with diet log records. The level of supplement intake is determined by bone density trends as measured using the monitors described here, diet logging (particularly of calcium level), or some combination of parameters. For a large number of users, diets successful in maintaining bone density can be identified, and these diets recommended to users.

Periodic professional testing may be used to monitor the bone density and other physiological parameters of the person.

Calcium rich foods and supplements are well known in the nutritional arts. For example, in U.S. Pat. No. 4,726,952, Pak describes the use of calcium citrate as a calcium supplement. Sodium fluoride is also identified as a supplement to enhance calcium uptake into bone material. Other calcium compounds which may be consumed as supplements include the various calcium phosphates, calcium stearate, calcium lactate, calcium gluconate, calcium chloride, calcium carbonate, dairy products, and other calcium-containing compounds and consumables.

Embodiments of the present invention may also be used with an activity sensor, as enhanced physical activity levels enhance bone strength. If activity levels are too low, the user can be warned, advised to consume supplements, or be referred to a fitness professional or the like for assisted activities. For example, a pedometer may be carried on the body of the person and communicate data to a PDA.

Health Management Business Model

A health management business supplies a customer (referred to as a user in this specification) with a bone density monitor. The user transfers data from the monitor to a computer having a link to a communications network such as the Internet. Data from the monitor is sent to a computer system associated with the business, analyzed, and feedback is then returned to the user. For example, if calcium supplements are required, the recommended level to be consumed is provided to the user. In one model, supplies of a calcium supplement are sent to the user automatically according to the levels recommended.

This business method can be adapted to other supplements. The health management business supplies a computer such as a PDA, or a software program to run on a computer such as a PDA, so that a user can record a diet log. The user records their diet log in terms of calories, fat, carbohydrates, protein, fiber, vitamins, minerals, and any other nutritional component of interest. Diet log data is then sent from the user over a communications network to a computer system associated with the health management business, such as a web server. The data is analyzed by employees or a computer expert system provided by the health management business, and supplies of nutritional supplements are provided as needed. For example, a person may receive a weekly package of supplements based on the diet log information that they supplied to the health management business. The supplements may be in the form of multiple tablet types, or a custom formulated capsule, beverage, powder, gel or other consumable item. In another embodiment, the health management business is in communication with a food retailer, and sends data to the food retailer concerning the nutritional needs of the user. For an on-line grocery store, the user is given feedback and specific suggestions of foods to eat when the user next orders food from the on-line grocery store.

The business may also supply feedback to the user. For example, if diet trends show low calcium and Vitamin A levels, feedback may be provided on foods which may be consumed to bring up the levels to a healthy recommended level. The Internet, interactive TV, printed materials, or any other convenient method may be used to provide feedback.

A business may also assist in formulating a shopping list for a user, e.g. adding calcium rich foods to a standing grocery order. This concept works well with on-line grocery store ordering. It may also be adapted to shopping at conventional grocery stores, for example using the generation of printed lists.

Purchase information may also be used in generating diet log information. For example, when a user purchases items at a grocery store, the product identities may be sent to a health management business which may then use this information in helping generate a diet log for the user. This is best described in a co-pending provisional application to James R. Mault, titled “Device for Diet Control and Shopping List Generation” (docket number MJA-22918) filed on Sep. 21, 2000.

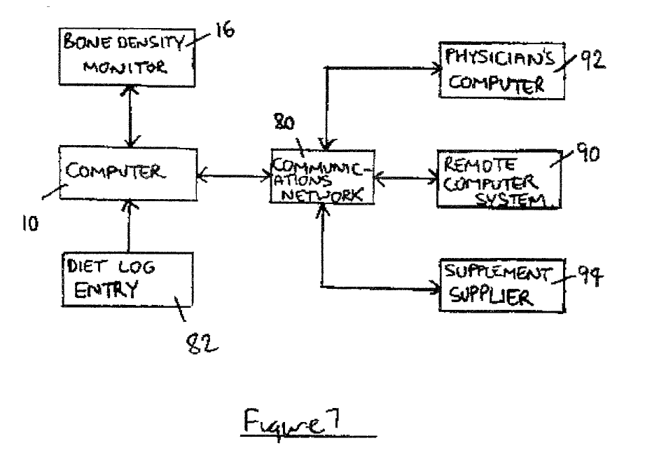

FIG. 7 shows a system embodiment. A bone density monitor 16 is in communication with computing device 10, the computing device 10 having a diet log entry mechanism 82. Computing device 10 is linked to a communications network 80, preferably the Internet. A remote computer system 90 provides a software program so as to receive diet log data, bone density data, and any other data of interest from computing device 10, and store it within the memory of system 90, for example in a database. The data stored within system 90 is accessible from physician's computer 92, for professional review. The data is analyzed by software running on remote computer system 90, so as to provide feedback to the user. Dietary supplements are sent to the user from a supplement supplier, having a computer system 94 with a link to the communications network so as to access user data stored on system 90. Bone density, dietary, and other physiological trends can be viewed by the user or other authorized person using a website generated by remote computer 90, or other computer in communication with it. Data can be updated or modified by interactions over the communications network. Data is synchronized over the communications network.

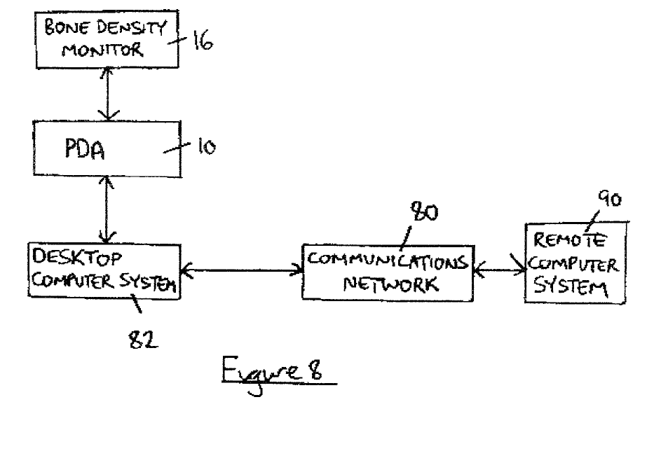

FIG. 8 shows a bone density monitor 16 in communication with a computing device 10, in this embodiment a PDA. The PDA 10 communicates with an additional desktop computing device 82. Desktop computing device 82 has a link to a communications network 80, and hence to remote computer system 90, as described above. A health management business may provide software to run on either PDA or desktop computer 82, so as to allow the user to engage in diet logging and analyze bone density data.

1. A method of health control comprising:
supporting an ultrasonic bone density monitor in contract with the body of a user to generate electrical signals representative of the user's bone density;
transmitting said electrical signals over a communications network to a remote computer system;
analyzing the bone density signals received at the remote computer system and generating feedback information for the user based upon said analysis; and
transmitting the feedback information to the user through the communications network and communicating the feedback information to the user.

2. The method of claim 1 wherein the ultrasonic monitor is supported on the body of the user.

3. The method of claim 1 wherein the ultrasonic monitor is incorporated in an appliance adapted to be placed in contact with the body of the user.

4. The method of claim 1 in which the ultrasonic monitor incorporates a removable memory and after use the memory is removed from the monitor and inserted into a reader for transmission to said remote computer.

5. The method of claim 1 wherein the transmission of the bone density information to remote computer includes providing the bone density information to a personal digital assistant and transmitting the information from the personal digital assistant to the remote computer system.

6. The method of claim 5 in which the feedback information to the user is displayed on a personal digital assistant.

7. The method of claim 1 in which the bone density monitor is adapted to be supported on the wrist of a user.

8. The method of claim 1 in which information as to the food consumption of the user is recorded and transmitted to the remote computer system.

9. The system of claim 8 wherein the feedback from the remote computer system includes information relating to desired diet changes by the user.

10. A system for monitoring a user's health condition based on bone density measurements, comprising:
an ultrasonic bone density monitor adapted to be brought into contact with the body of the user to produce electrical signals representative of the user's bone density;
means for storing and displaying said bone density signals;
a remote computer system containing a program for analyzing bone density information and generating feedback reports based upon said analysis;
a communications network connecting said means for recording and displaying bone density information to the remote computer system; and
means for transmitting feedback reports from the remote transmitter system to the means for recording and displaying bone density information.

11. The system of claim 1 wherein the bone density monitor employs a pair of spaced ultrasonic transducers.

12. The system of claim 10 wherein the means for recording and displaying the output of the bone density monitor comprises a personal digital assistant.

13. The system of claim 10 wherein the bone density monitor includes a removable memory and information detected by the bone density monitor is provided to a personal digital assistant by removing memory and inserting the memory in a port of the personal digital assistant.

14. The system of claim 10 further including means for recording the user's food and drink consumption and transmitting the information to the remote computer system for analysis.

15. The system of claim 13 wherein the feedback information from the remote computer system includes diet recommendations for the user.
